(19) United States
(12) Patent Application Publication
(10) Pub. No.: US 2024/0148333 A1
(43) Pub. Date: May 9, 2024

(54) ESTIMATION APPARATUS, ESTIMATION METHOD, AND NON-TRANSITORY COMPUTER-READABLE RECORDING MEDIUM

(71) Applicant: NEC Corporation, Tokyo (JP)

(72) Inventors: Chenhui Huang, Tokyo (JP); Kenichiro Fukushi, Tokyo (JP); Yusuke Sekiguchi, Sendai-shi (JP); Haruki Yaguchi, Sendai-shi (JP); Keita Honda, Sendai-shi (JP); Shinichi Izumi, Sendai-shi (JP); Dai Owaki, Sendai-shi (JP)

(73) Assignee: NEC Corporation, Tokyo (JP)

(21) Appl. No.: 18/413,102

(22) Filed: Jan. 16, 2024

Related U.S. Application Data
(63) Continuation of application No. 17/362,351, filed on Jun. 29, 2021.

(30) Foreign Application Priority Data
Jul. 3, 2020 (JP) 2020-115936

(51) Int. Cl. A61B 5/00; A61B 5/103; G01D 5/12; G16H 40/67; G16H 50/30

(52) U.S. Cl. CPC A61B 5/7275; A61B 5/1038; A61B 5/6807; G01D 5/12; G16H 40/67; G16H 50/30

(57) ABSTRACT

An estimation apparatus acquires stance phases of both feet and estimates a risk of abnormality of a lower limb on the basis of the difference between the stance phases of the both feet.

### CROSS-REFERENCE TO RELATED APPLICATIONS

This application is a Continuation of U.S. application Ser. No. 17/362,351, filed Jun. 29, 2021, which is based upon and claims the benefit of priority from Japanese Patent Application No. 2020-115936, filed Jul. 3, 2020, the disclosure of which is incorporated herein in its entirety by reference.

### TECHNICAL FIELD

The present invention relates to an estimation apparatus, an estimation method, and a non-transitory computer-readable recording medium.

### BACKGROUND ART

For health management, changes in motions of lower limbs are monitored to determine abnormalities in the lower limbs. As related arts, the techniques of Japanese Patent No. 4350394 (hereinafter referred to as “Patent Document 1”) and Japanese Patent No. 5586050 (hereinafter referred to as “Patent Document 2”) are disclosed. Patent Document 1 discloses a technique in which an acceleration sensor is worn on the tibial portion and an abnormality in the lower limbs is estimated from a waveform power spectrum. Moreover, Patent Document 2 discloses a technique in which a plurality of sensor units are attached to each of both feet and synchronized waveforms of both feet are measured.

### SUMMARY

In recent years, it has been reported that the bone density of the lower limb on the side with a shorter stance phase decreases due to a gap between periods in which the left and right lower limbs contact the ground (stance phases) during walking. Decreased bone density causes bone fragility and can lead to fractures easily forming. In addition, such a gap indicates a decrease in balance ability. It is desired to detect disturbances caused by such a gap.

Therefore, an example object of the present invention is to provide an estimation apparatus, an estimation method, and a non-transitory computer-readable recording medium that solves the above-described problems.

A first example aspect of the present invention is an estimation apparatus including: at least one memory configured to store instructions; and at least one processor configured to execute the instructions to: acquire stance phases of both feet; and estimate a risk of abnormality of a lower limb on the basis of a difference between the stance phases of the both feet.

A second example aspect of the present invention is an estimation method including: acquiring stance phases for both feet, and estimating a risk of abnormality of a lower limb on the basis of a difference between the stance phases of the both feet.

A third example aspect of the present invention is a non-transitory computer readable recording medium that records a program for causing a computer of an estimation apparatus to execute: acquiring stance phases of both feet; and estimating a risk of abnormality of a lower limb on the basis of a difference between the stance phases of the both feet.

According to the present invention, it is possible to estimate the risk of abnormality of the lower limb based on the difference between the left and right stance phases during a walking motion.

### BRIEF DESCRIPTION OF THE DRAWINGS

FIG. 1 is a diagram showing a schematic configuration of a lower limb abnormality risk determination system according to an example embodiment of the present invention.

FIG. 2 is a diagram showing a hardware configuration of an estimation apparatus, a first sensor, and a second sensor according to an example embodiment of the present invention.

FIG. 3 is a functional block diagram of the estimation apparatus, the first sensor, and the second sensor according to a first example embodiment.

FIG. 4 is a first diagram illustrating a stance phase according to an example embodiment of the present invention.

FIG. 5 is a second diagram illustrating the stance phase according to an example embodiment of the present invention.

FIG. 6 is a diagram showing an example of usage of the estimation apparatus, the first sensor, and the second sensor according to an example embodiment of the present invention.

FIG. 7 is a diagram showing an outline of a sensor apparatus provided in a shoe sole according to an example embodiment of the present invention.

FIG. 8 is a diagram showing the processing flow of each apparatus in the lower limb abnormality risk determination system according to the first example embodiment.

FIG. 9 is a functional block diagram of the estimation apparatus, the first sensor, and the second sensor according to a second example embodiment.

FIG. 10 is a diagram showing the processing flow of each apparatus in the lower limb abnormality risk determination system according to the second example embodiment.

FIG. 11A is a diagram showing the plantar flexion state of a foot according to an example embodiment of the present invention.

FIG. 11B is a diagram showing the dorsiflexion state of a foot according to an example embodiment of the present invention.

FIG. 12 is a diagram showing an outline of calculation of the stance phase according to a third example embodiment.

FIG. 13 is a diagram showing an outline of the sensor apparatus provided on a shoe sole according to a fourth example embodiment.

FIG. 14 is a diagram showing an outline of calculation of the stance phase according to the fourth example embodiment.

FIG. 15 is a diagram showing a schematic configuration of a lower limb abnormality risk determination system according to another example embodiment.

FIG. 16 is a diagram showing the minimum configuration of the estimation apparatus.

FIG. 17 is a diagram showing a processing flow of the estimation apparatus with the minimum configuration.

### EXAMPLE EMBODIMENT

Hereinbelow, a lower limb abnormality risk determination system according to an example embodiment of the present invention will be described with reference to the drawings.

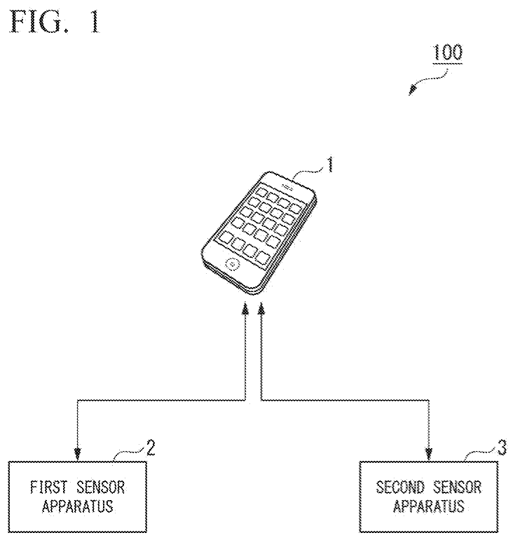

FIG. 1 is a diagram showing a schematic configuration of a lower limb abnormality risk determination system according to the present example embodiment.

As shown in FIG. 1, the lower limb abnormality risk determination system 100 is configure by at least an estimation apparatus 1, a first sensor apparatus 2, and a second sensor apparatus 3. The estimation apparatus 1 connects to the first sensor apparatus 2 and the second sensor apparatus 3 and communicates with the first sensor apparatus 2 and the second sensor apparatus 3 in order to acquire sensing information detected by each of the sensors of the first sensor apparatus 2 and the second sensor apparatus 3.

The first sensor apparatus 2 and the second sensor apparatus 3 are attached to the sole of a left shoe and the sole of a right shoe, respectively, measure the acceleration or angular velocity of the respective foot, and transmit to the estimation apparatus 1 information on the stance phase of each foot calculated on the basis of the acceleration or angular velocity. The estimation apparatus 1 receives the information on the stance phase from each of the first sensor apparatus 2 and the second sensor apparatus 3, and determines a risk of abnormality of a lower limb (hereinafter referred to as “a lower limb abnormality risk”) on the basis of the information of the stance phases.

The estimation apparatus 1 may be a mobile terminal such as a smartphone. Moreover, the estimation apparatus 1 may be any apparatus as long as it can receive sensing information from the first sensor apparatus 2 and the second sensor apparatus 3 and determine the lower limb abnormality risk. For example, the estimation apparatus 1 may be a server apparatus provided remotely.

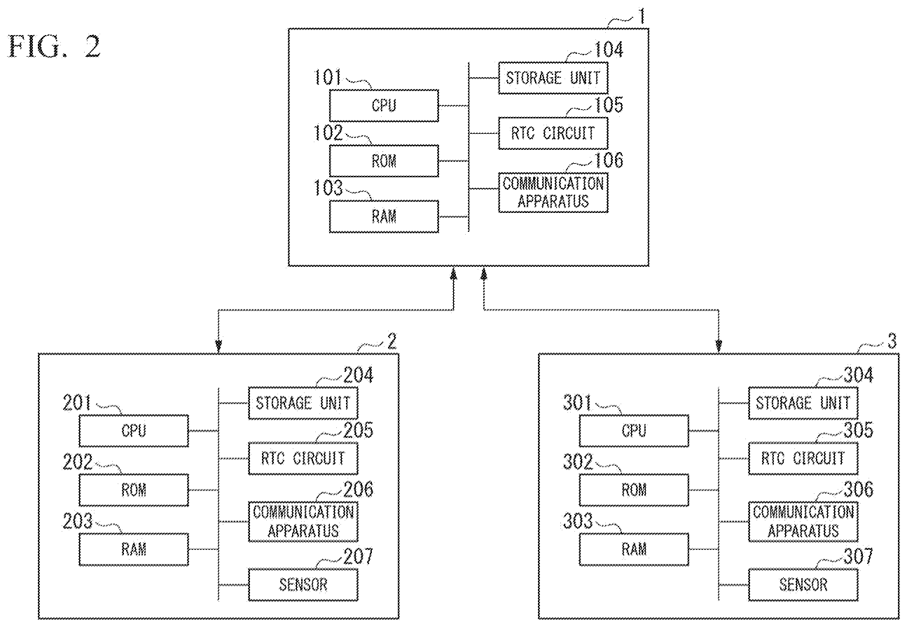

FIG. 2 is a diagram showing a hardware configuration of the estimation apparatus, the first sensor, and the second sensor.

The estimation apparatus 1 is a computer that includes hardware such as a central processing unit (CPU) 101, a read only memory (ROM) 102, a random access memory (RAM) 103, a storage unit 104, a real time clock (RTC) circuit 105, and a communication apparatus 106.

Moreover, the first sensor apparatus 2 is a computer that includes hardware such as a CPU 201, a ROM 202, a RAM 203, a storage unit 204, an RTC circuit 205, a communication apparatus 206, and a sensor 207.

Moreover, the second sensor apparatus 3 is a computer that includes hardware such as a CPU 301, a ROM 302, a RAM 303, a storage unit 304, an RTC circuit 305, a communication apparatus 306, and a sensor 307.

In the present example embodiment, the sensor 207 of the first sensor apparatus 2 and the sensor 307 of the second sensor apparatus 3 are each configured by inertial measurement units (IMU) that sense an acceleration and/or an angular velocity based on a motion of a foot when a user walks.

### First Example Embodiment

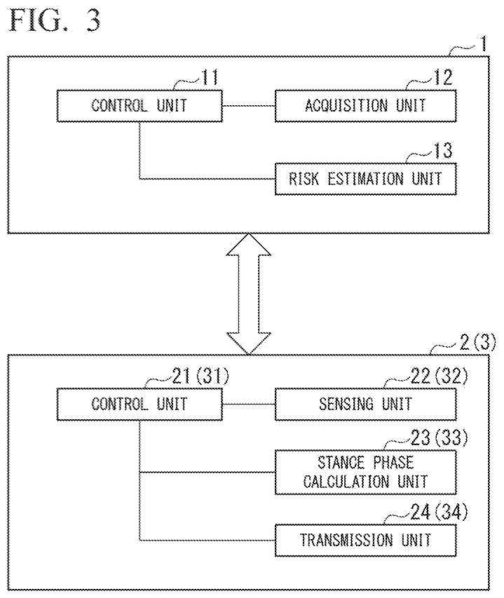

FIG. 3 is a functional block diagram of the estimation apparatus, the first sensor, and the second sensor according to the first example embodiment.

The estimation apparatus 1 executes a lower limb abnormality risk determination program stored in advance. Thereby, the estimation apparatus 1 exerts the functions of at least a control unit 11, an acquisition unit 12, and a risk estimation unit 13.

The control unit 11 of the estimation apparatus 1 controls other functional units of the estimation apparatus 1.

The acquisition unit 12 of the estimation apparatus 1 acquires information on the stance phase of each of both feet.

The risk estimation unit 13 of the estimation apparatus 1 estimates the lower limb abnormality risk on the basis of the difference between the stance phases of both feet.

Moreover, the first sensor apparatus 2 executes a sensing program stored in advance. Thereby, the first sensor apparatus 2 exerts the functions of at least a control unit 21, a sensing unit 22, a stance phase calculation unit 23, and a transmission unit 24.

The control unit 21 of the first sensor apparatus 2 controls other functional units of the first sensor apparatus 2.

The sensing unit 22 of the first sensor apparatus 2 acquires from the sensor 207 of the IMU or the like the acceleration and/or the angular velocity based on the motion of the left foot when the user walks.

The stance phase calculation unit 23 detects the stance phase of the left foot on the basis of the acceleration and/or the angular velocity of the left foot.

The transmission unit 24 of the first sensor apparatus 2 transmits information on the stance phase of the left foot to the estimation apparatus 1.

Moreover, the second sensor apparatus 3 executes a sensing program stored in advance. Thereby, the second sensor apparatus 3 exerts the functions of at least a control unit 31, a sensing unit 32, a stance phase calculation unit 33, and a transmission unit 34.

The control unit 31 of the second sensor apparatus 3 controls other functional units of the second sensor apparatus 3.

The sensing unit 32 of the second sensor apparatus 3 acquires from the sensor 307 of the IMU or the like the acceleration and/or the angular velocity based on the motion of the right foot when the user walks.

The stance phase calculation unit 33 detects the stance phase of the right foot on the basis of the acceleration and/or the angular velocity of the right foot.

The transmission unit 34 of the second sensor apparatus 3 transmits information on the stance phase of the right foot to the estimation apparatus 1.

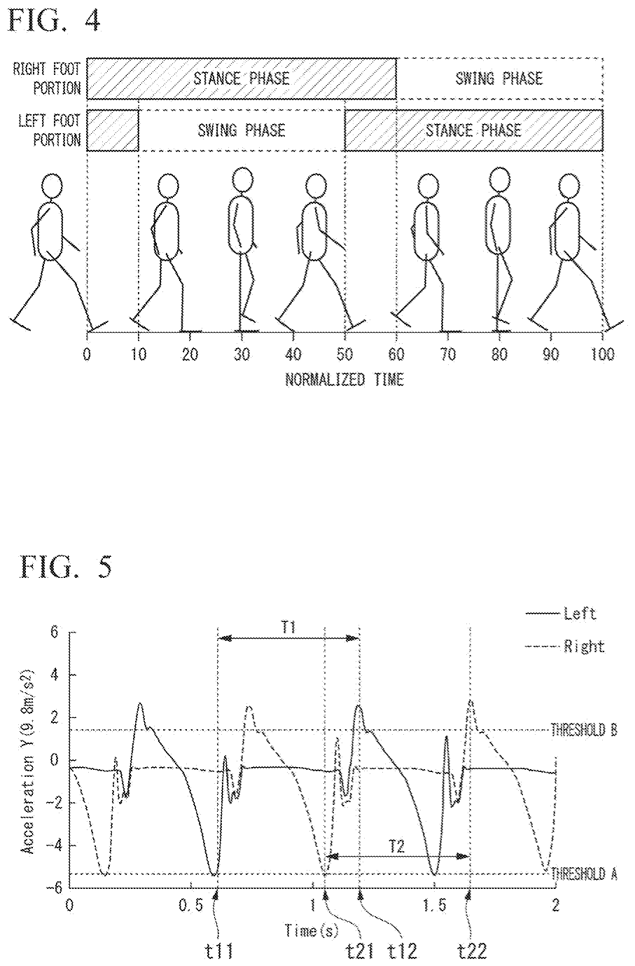

FIG. 4 is a first diagram illustrating a walking period.

FIG. 4 is a diagram showing a stance phase and a swing phase of the left foot and the right foot in the walking motion of a person. One cycle of the motion cycle of the walking motion is represented by 0% to 100%, with the time at which the heel of one foot lands being 0%, and the time at which the heel of the same foot lands next being 100%. In the motion cycle of this walking motion, the period from the time when the heel of the right foot lands to the time when the toe of the right foot takes off is called the stance phase of the right foot, and the period from the time when the heel of the left foot lands to the time when the toe of the left foot takes off is called the stance phase of the left foot.

FIG. 5 is a second diagram illustrating the stance phase.

FIG. 5 shows acceleration on the vertical axis in the motion cycle of the walking motion of the left foot and the right foot. In FIG. 5, the horizontal axis represents time and the vertical axis represents acceleration. It should be noted that the negative acceleration indicates a downward acceleration, and the positive acceleration indicates an upward acceleration. Moreover, in FIG. 5, the solid line shows the transition of the acceleration of the left foot, and the dotted line shows the transition of the acceleration of the right foot.

Time t11 indicates the timing immediately before the left foot lands, and time t12 indicates the timing immediately after the left foot takes off. At the timing immediately before landing at time t11, the downward acceleration exceeds a threshold A and peaks, and at the timing immediately after takeoff at time t12, the upward acceleration exceeds a threshold B and peaks. In the present example embodiment, the first sensor apparatus 2 detects the time t11 and the time t12, and calculates the stance phase of the left foot on the basis of the time difference therebetween. The first sensor apparatus 2 may calculate a statistical value such as the average of the time difference between the time t11 and the time t12 in each motion cycle of the walking motion as the stance phase of the left foot.

Moreover, time t21 indicates the timing immediately before the right foot lands, and time t22 indicates the timing immediately after the right foot takes off. At the timing immediately before landing at time t21, the downward acceleration exceeds the threshold A and peaks, and at the timing immediately after takeoff at time t22, the upward acceleration exceeds the threshold B and peaks. In the present example embodiment, the second sensor apparatus 3 detects the time t21 and the time t22, and calculates the stance phase of the right foot on the basis of the time difference therebetween. The second sensor apparatus 3 may calculate a statistical value such as the average of the time difference between the time t21 and the time t22 in each motion cycle of the walking motion as the stance phase of the right foot.

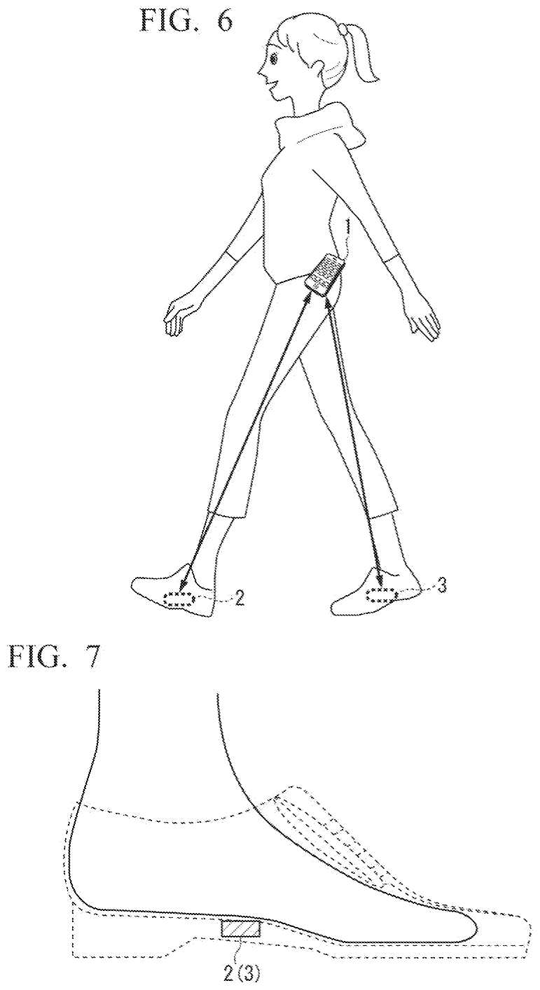

FIG. 6 is a diagram showing an example of usage of the estimation apparatus, the first sensor, and the second sensor.

As an example, the estimation apparatus 1 is carried by the user. Then, the first sensor apparatus 2 is mounted in the insole of the shoe of the left foot and near the arch of the left foot of the user. Moreover, the second sensor apparatus 3 is mounted in the insole of the shoe of the right foot and near the arch of the right foot of the user. Then, the first sensor apparatus 2 and the second sensor apparatus 3 each calculate the stance phase on the basis of the acceleration and/or the angular velocity detected in accordance with the motion of each respective foot due to the walking of the user, and transmit the calculated stance phase to the estimation apparatus 1.

FIG. 7 is a diagram showing an outline of the sensor apparatus provided in a shoe sole.

As shown in FIG. 7, the first sensor apparatus 2 and the second sensor apparatus 3 are respectively provided in the soles of the shoes. The first sensor apparatus 2 and the second sensor apparatus 3 connect to the estimation apparatus 1 and communicate with the estimation apparatus 1 through wireless communication.

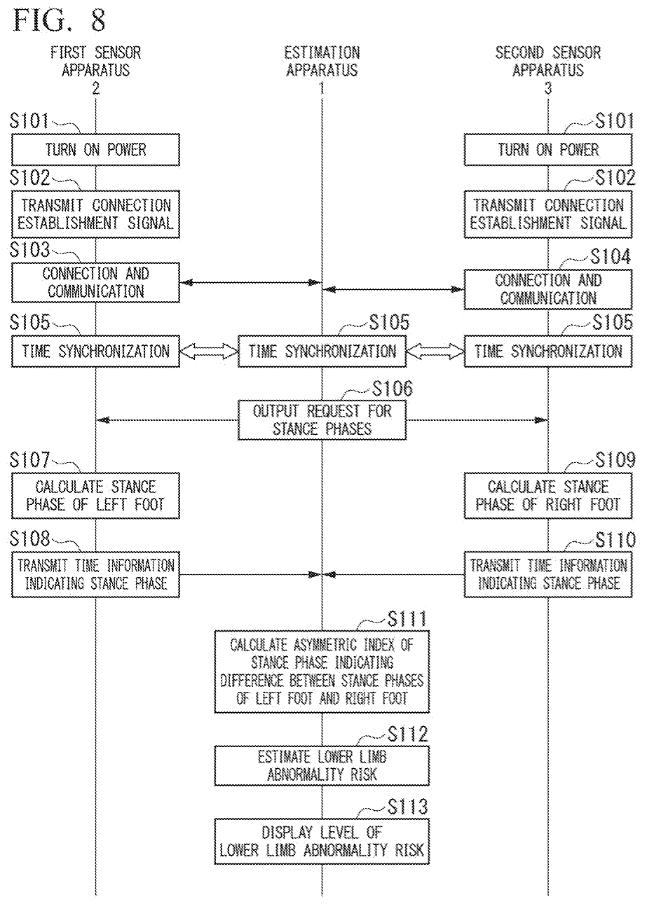

FIG. 8 is a diagram showing the processing flow of each apparatus in the lower limb abnormality risk determination system according to the first example embodiment. The user turns on the power of the first sensor apparatus 2 and the second sensor apparatus 3 (step S101). Thereby, the communication apparatus 206 of the first sensor apparatus 2 and the communication apparatus 306 of the second sensor apparatus 3 transmit connection establishment signals (step S102). These communication apparatuses 206 and 306 each have a wireless communication function such as Bluetooth Low Energy (BLE; registered trademark) and WiFi (registered trademark) as an example, and use this function to connect to other apparatuses and communicate with the other apparatuses.

The user operates the estimation apparatus 1 to allow connection to the first sensor apparatus 2 and communication with the first sensor apparatus 2. Thereby, the estimation apparatus 1 connects to the first sensor apparatus 2 and communicates with the first sensor apparatus 2 (step S103). Similarly, the user operates the estimation apparatus 1 to allow connection to the second sensor apparatus 3 and communication with the second sensor apparatus 3. Thereby, the estimation apparatus 1 connects to the second sensor apparatus 3 and communicates with the second sensor apparatus 3 (step S104). The user instructs the estimation apparatus 1 to start processing. Then, the control unit 11 of the estimation apparatus 1 synchronizes the time between the first sensor apparatus 2 and the second sensor apparatus 3 (step S105). Thereby, the times measured by the first sensor apparatus 2, the second sensor apparatus 3, and the estimation apparatus 1 coincide with each other. That is, the control unit 11 of the estimation apparatus 1 has a function of a time synchronization processing unit. The control unit 11 transmits an output request for the stance phase of the left foot to the first sensor apparatus 2, and transmits an output request for the stance phase of the right foot to the second sensor apparatus 3 (step S106).

In the first sensor apparatus 2, the sensing unit 22 acquires the acceleration from the sensor 207. The stance phase calculation unit 23 calculates the stance phase of the left foot (step S107). Then, the transmission unit 24 transmits the time information indicating the stance phase of the left foot to the estimation apparatus 1 (step S108). The first sensor apparatus 2 may transmit the time information indicated by each stance phase of the left foot calculated for each motion cycle of the walking motion of the user to the estimation apparatus 1. Similarly, in the second sensor apparatus 3, the sensing unit 32 acquires the acceleration from the sensor 307. The stance phase calculation unit 33 calculates the stance phase of the right foot (step S109). Then, the transmission unit 34 transmits the time information indicating the stance phase of the right foot to the estimation apparatus 1 (step S110). The second sensor apparatus 3 may transmit the time information indicated by each stance phase of the right foot calculated for each motion cycle of the walking motion of the user to the estimation apparatus 1. Upon having calculated the statistical information of the stance phase in a predetermined period such as 1 minute, the first sensor apparatus 2 and the second sensor apparatus 3 may transmit those pieces of statistical information to the estimation apparatus 1 over a plurality of times.

The estimation apparatus 1 receives the time information of the stance phase of the left foot from the first sensor apparatus 2. Moreover, the estimation apparatus 1 receives the time information of the stance phase of the right foot from the second sensor apparatus 3. Then, the acquisition unit 12 of the estimation apparatus 1 acquires the time information of the stance phase of the left foot and the time information of the stance phase of the right foot. The risk estimation unit 13 of the estimation apparatus 1 acquires time information of a plurality of stance phases from the first sensor apparatus 2 and the second sensor apparatus 3. The risk estimation unit 13 calculates the time average of the plurality of stance phases of the left foot acquired from the first sensor apparatus 2 as the time of the stance phase of the left foot. Moreover, the risk estimation unit 13 calculates the time average of the plurality of stance phases of the right foot acquired from the second sensor apparatus 3 as the time of the stance phase of the right foot. The risk estimation unit 13 calculates an asymmetry index of stance phases indicating the difference between the time of stance phase of the left foot and the time of the stance phase the right foot (step S111). The risk estimation unit 13 estimates the lower limb abnormality risk on the basis of the asymmetry index of stance phases indicating the difference between the time of the stance phase of the left foot and the time of the stance phase of the right foot (step S112).

Specifically, the risk estimation unit 13 identifies a risk level on the basis of the time length indicated by the asymmetry index and a threshold. For example, the risk estimation unit 13 compares a time T indicated by the asymmetry index with thresholds a, b, c, and d (0<a<b<c<d). When the time T of the asymmetry index satisfies a≤T<b, the risk estimation unit 13 identifies the risk level as Level 1. When the time T of the asymmetry index satisfies b≤T<c, the risk estimation unit 13 identifies the risk level as Level 2. When the time T of the asymmetry index satisfies c≤T<d, the risk estimation unit 13 identifies the risk level as Level 3. If the estimation apparatus 1 is, for example, a smartphone, the risk estimation unit 13 displays the identified level of the lower limb abnormality risk on the liquid crystal display of the smartphone (step S113).

The risk estimation unit 13 may identify the level of the lower limb abnormality risk by another technique. For example, the risk estimation unit 13 may perform machine learning using the relationship between the length of time indicated by the asymmetry index and the actual occurrence event of a disturbance and/or the level of a disturbance, and calculate the level of the lower limb abnormality risk and/or a disturbance in accordance with the time indicated by the asymmetry index.

The first example embodiment of the lower limb abnormality risk determination system has been described above. According to the above-mentioned processing, it is possible to estimate the lower limb abnormality risk on the basis of the left and right stance phases.

### Second Example Embodiment

In the above processing, each sensor apparatus calculates the stance phase of the corresponding foot. However, the estimation apparatus 1 may calculate the stance phase. Hereinbelow, an example embodiment in which the estimation apparatus 1 calculates the stance phase will be described.

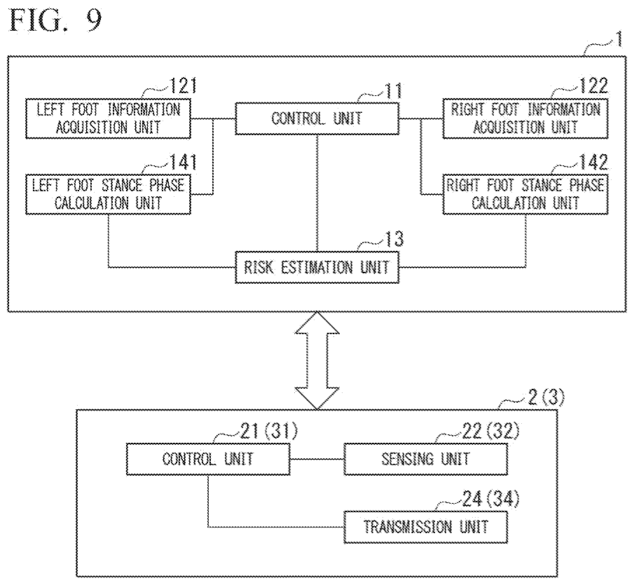

FIG. 9 is a functional block diagram of the estimation apparatus, the first sensor, and the second sensor according to the second example embodiment.

In the lower limb abnormality risk determination system according to the second example embodiment, the estimation apparatus 1 includes a control unit 11, a left foot information acquisition unit 121, a right foot information acquisition unit 122, a left foot stance phase calculation unit 141, a right foot stance phase calculation unit 142, and a risk estimation unit 13.

The left foot information acquisition unit 121 acquires sensing information from the first sensor apparatus 2.

The right foot information acquisition unit 122 acquires sensing information from the second sensor apparatus 3.

The left foot stance phase calculation unit 141 calculates the stance phase of the left foot.

The right foot stance phase calculation unit 142 calculates the stance phase of the right foot.

The risk estimation unit 13 estimates the lower limb abnormality risk as in the first example embodiment.

Moreover, in the lower limb abnormality risk determination system according to the second example embodiment, the first sensor apparatus 2 and the second sensor apparatus 3 each do not include a stance phase calculation unit, the first sensor apparatus 2 includes the control unit 21, the sensing unit 22, and the transmission unit 24, and the second sensor apparatus 3 includes the control unit 31, the sensing unit 32, and the transmission unit 34.

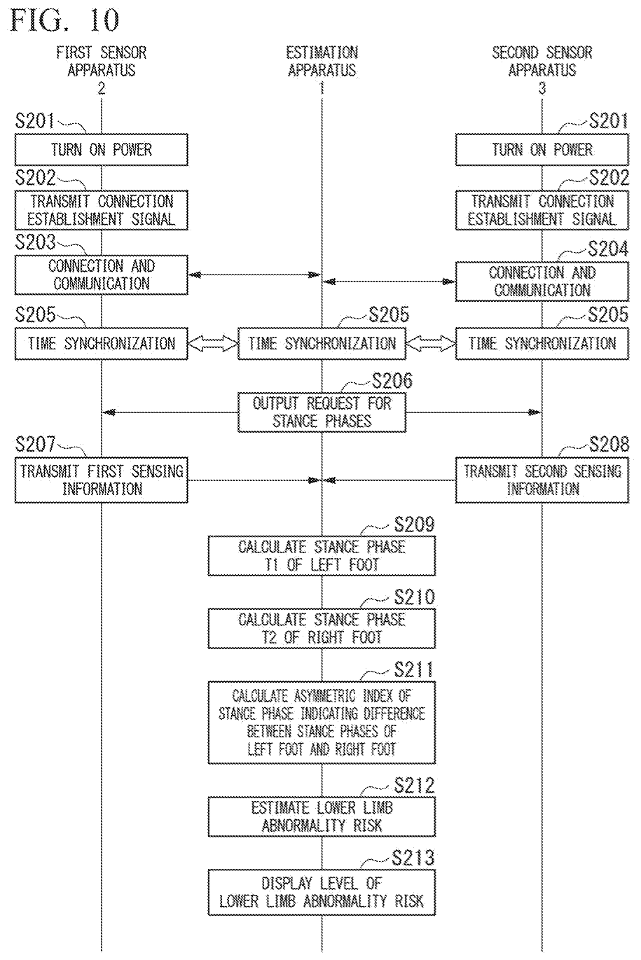

FIG. 10 is a diagram showing the processing flow of each apparatus in the lower limb abnormality risk determination system according to the second example embodiment.

Similarly to the first example embodiment, the user turns on the power of the first sensor apparatus 2 and the second sensor apparatus 3 (step S201). Thereby, the communication apparatus 206 of the first sensor apparatus 2 and the communication apparatus 306 of the second sensor apparatus 3 transmit connection establishment signals (step S202). These communication apparatuses 206 and 306 have a wireless communication function such as Bluetooth Low Energy (BLE; registered trademark) and WiFi (registered trademark) as an example, and use this function to connect to other apparatuses and communicate with the other apparatuses.

The user operates the estimation apparatus 1 to allow connection to the first sensor apparatus 2 and communication with the first sensor apparatus 2. Thereby, the estimation apparatus 1 connects to the first sensor apparatus 2 and communicates with the first sensor apparatus 2 (step S203). Similarly, the user operates the estimation apparatus 1 to allow connection to the second sensor apparatus 3 and communication with the second sensor apparatus 3. Thereby, the estimation apparatus 1 connects to the second sensor apparatus 3 and communicates with the second sensor apparatus 3 (step S204). The user instructs the estimation apparatus 1 to start processing. Then, the control unit 11 of the estimation apparatus 1 synchronizes the time between the first sensor apparatus 2 and the second sensor apparatus 3 (step S205). Thereby, the times measured by the first sensor apparatus 2, the second sensor apparatus 3, and the estimation apparatus 1 coincide with each other. That is, the control unit 11 of the estimation apparatus 1 has a function of a time synchronization processing unit. The control unit 11 transmits an output request for the stance phase of the left foot to the first sensor apparatus 2, and transmits an output request for the stance phase of the right foot to the second sensor apparatus 3 (step S206).

The first sensor apparatus 2 repeatedly transmits first sensing information including at least the acceleration of the left foot to the estimation apparatus 1 at a predetermined interval on the basis of the output request from the estimation apparatus 1 (step S207). Similarly, the second sensor apparatus 3 repeatedly transmits second sensing information including at least the acceleration of the right foot to the estimation apparatus 1 at a predetermined interval on the basis of the output request from the estimation apparatus 1 (step S208). The estimation apparatus 1 receives the first sensing information transmitted from the first sensor apparatus 2 and the second sensing information transmitted from the second sensor apparatus 3.

The left foot information acquisition unit 121 repeatedly acquires the first sensing information and outputs the acquired first sensing information to the left foot stance phase calculation unit 141. Moreover, the right foot information acquisition unit 122 repeatedly acquires the second sensing information and outputs the acquired second sensing information to the right foot stance phase calculation unit 142.

The left foot stance phase calculation unit 141 sequentially compares the acceleration values included in the repeatedly acquired first sensing information, and identifies the time t11 at which the downward acceleration (negative acceleration) in the direction of lowering the foot exceeds the threshold A and shows a peak. The left foot stance phase calculation unit 141 sequentially compares the acceleration values included in the repeatedly acquired first sensing information, and identifies the time t12 at which the upward acceleration (positive acceleration) in the direction of raising the foot exceeds the threshold B and shows a peak. The left foot stance phase calculation unit 141 calculates a stance phase T1 of the left foot indicating the difference between the time t11 and the time t12 (step S209). Similarly, the left foot stance phase calculation unit 141 calculates the stance phase T1 of the left foot for each motion cycle of the walking motion of the user. The left foot stance phase calculation unit 141 sequentially outputs the calculated stance phase T1 of the left foot to the risk estimation unit 13.

Similarly, the right foot stance phase calculation unit 142 sequentially compares the acceleration values included in the repeatedly acquired second sensing information, and identifies the time t21 when the downward acceleration (negative acceleration) in the direction of lowering the foot exceeds the threshold A and shows a peak. The right foot stance phase calculation unit 142 sequentially compares the acceleration values included in the repeatedly acquired second sensing information, and identifies the time t22 when the upward acceleration (positive acceleration) in the direction of raising the foot exceeds the threshold B and shows a peak. The right foot stance phase calculation unit 142 calculates a stance phase T2 of the right foot indicating the difference between the time t21 and the time t22 (step S210). Similarly, the right foot stance phase calculation unit 142 calculates the stance phase T2 of the right foot for each motion cycle of the walking motion of the user. The right foot stance phase calculation unit 142 sequentially outputs the calculated stance phase T2 of the right foot to the risk estimation unit 13.

The risk estimation unit 13 calculates the average of the times of a plurality of stance phases T1 of the left foot acquired from the left foot stance phase calculation unit 141 as the time of the stance phase of the left foot. Moreover, the risk estimation unit 13 calculates the average of the times of the plurality of stance phases T2 of the right foot acquired from the right foot stance phase calculation unit 142 as the time of the stance phase of the right foot. The risk estimation unit 13 calculates an asymmetry index of stance phases indicating the difference between the time of the stance phase of the left foot and the time of the stance phase of the right foot. The risk estimation unit 13 estimates the lower limb abnormality risk on the basis of the asymmetry index of stance phases, which indicates the difference between the time of the stance phase of the left foot and the time of the stance phase of the right foot. A specific example of this estimation is the same as the processing of the first example embodiment.

That is, the risk estimation unit 13 calculates an asymmetry index of stance phases indicating the difference between the time of the stance phase of the left foot and the time of the stance phase of the right foot (step S211). The risk estimation unit 13 estimates the lower limb abnormality risk on the basis of the asymmetry index of stance phases indicating the difference between the time of the stance phase of the left foot and the time of the stance phase of the right foot (step S212). Then, if the estimation apparatus 1 is a smartphone, the risk estimation unit 13 displays the identified level of the lower limb abnormality risk on the liquid crystal display of the smartphone, as in the first example embodiment described above (step S213).

### Third Example Embodiment

The first sensor apparatus 2 and the second sensor apparatus 3 can sense acceleration and angular velocity as described above. In this case, the angle of a foot can be calculated using the acceleration and the angular velocity. More specifically, the estimation apparatus 1 acquires the lateral acceleration, vertical acceleration, antero-posterior acceleration of a foot, the vertical rotational angular velocity of the foot portion, the lateral rotational angular velocity of the foot portion, and the internal-external rotational angular velocity of the foot portion included in the sensing information. It should be noted that when the plane of the foot sole and the leg are perpendicular, the axis connecting the heel and the toe is denoted as the first axis, the axis parallel to the leg and passing through the ankle is denoted as the second axis, and the axis perpendicular to the first axis and the second axis is denoted as the third axis. In this case, the angular velocity of rotation around the third axis is called the vertical rotational angular velocity of the foot portion. Moreover, the angular velocity of rotation around the second axis is called the lateral rotational angular velocity of the foot portion. Furthermore, the angular velocity of rotation around the first axis is called the internal-external rotational angular velocity of the foot portion. Then, the estimation apparatus 1 uses an angle calculation program to calculate the vertical rotational angle of the foot portion indicating the angle around the third axis, the lateral rotational angle indicating the angle around the second axis, and the internal-external rotational angle indicating the angle around the third axis. For example, a Madgwick filter is known as the angle calculation program, and a known technique may be used. The estimation apparatus 1 calculates the stance phase by using the vertical rotational angle among the vertical rotational angle, the lateral rotational angle, and the internal-external rotational angle.

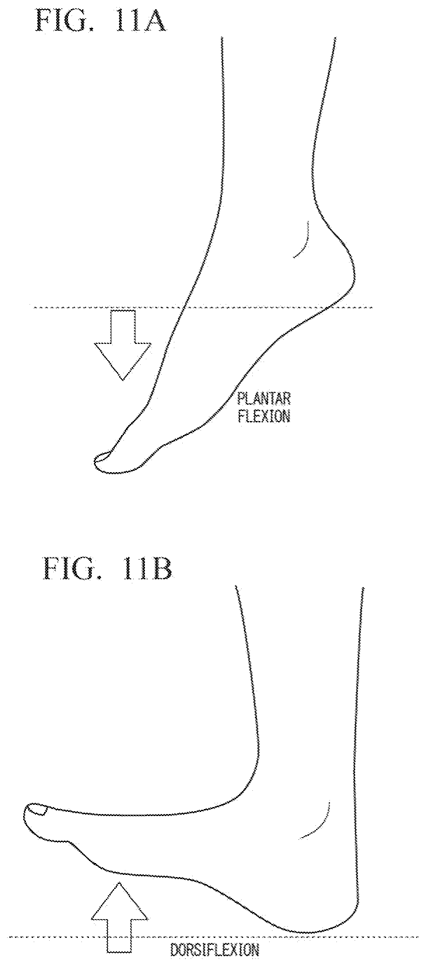

FIGS. 11A and 11B are diagrams showing a plantar flexion state and a dorsiflexion state of a foot portion, respectively.

That is, at the timing of the landing of the heel in the walking motion, the first axis connecting the heel and the toe is in a dorsiflexion state in which the toe is raised with the ankle serving as a fulcrum (FIG. 11B). Moreover, at the timing of taking off in the walking motion, the first axis is in a plantar flexion state in which the toe is lowered with the ankle as a fulcrum (FIG. 11A). However, due to disease, each timing in the stance phase does not always coincide with the time point at which the maximum dorsiflexion angle and the maximum plantar flexion angle are reached. In order to address such a problem, the stance phase calculation unit 23 (33) creates, by means of machine learning, an estimation model of the relationship between the timings of start/end of the stance phase and the angle of the ankle joint during walking, which is measured in advance, and estimates start/end of the stance phase by inputting angle information at the time of the measurement to the estimation model. This solves the above problem.

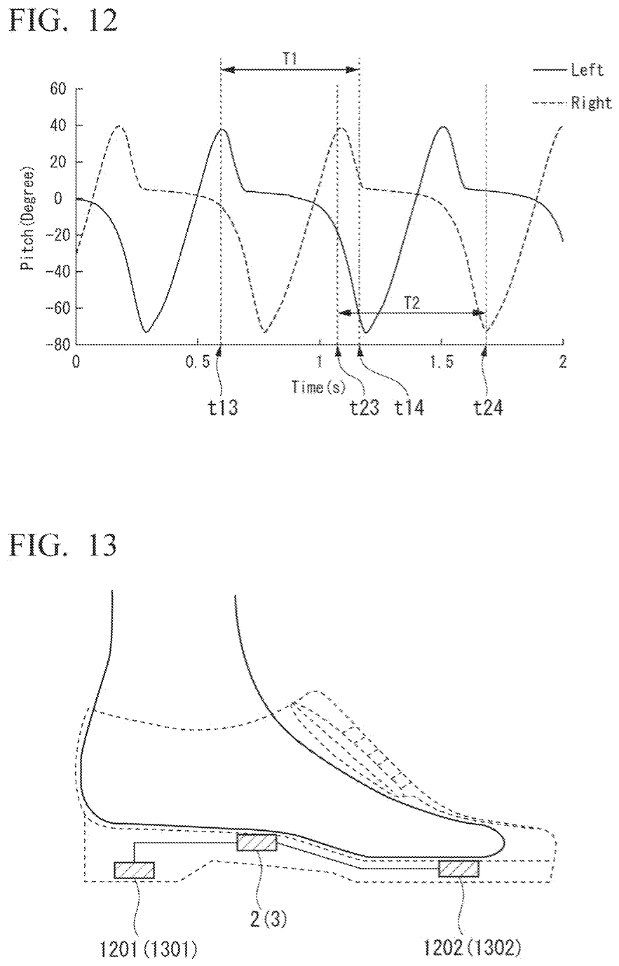

FIG. 12 is a diagram showing an outline of calculation of the stance phase according to the third example embodiment.

FIG. 12 shows the vertical rotational angle of the foot portion in the motion cycle of the walking motion of the left foot and the right foot. In FIG. 12, the horizontal axis represents time and the vertical axis represents the vertical rotational angle. It should be noted that a positive vertical rotational angle indicates a dorsiflexion state, and a negative vertical rotational angle indicates a plantar flexion state. Moreover, in FIG. 12, the solid line shows the transition of the vertical rotational angle of the left foot, and the dashed line shows the transition of the vertical rotational angle of the right foot.

Time t13 indicates the timing immediately before the left foot lands, and time t14 indicates the timing immediately after the left foot takes off. At the timing immediately before landing at time t13, the vertical rotational angle has the largest peak in the positive direction, and at the timing immediately after takeoff at time t14, the vertical rotational angle has the largest peak in the negative direction. Then, the estimation apparatus 1 repeatedly calculates the vertical rotational angle at short intervals on the basis of the first sensing information, and calculates the difference between the time t13 and the time t14 as the stance phase of the left foot. The estimation apparatus 1 may calculate a statistical value such as the average of the time differences between the time t13 and the time t14 in each motion cycle of the walking motion as the stance phase of the left foot. Moreover, the estimation apparatus 1 repeatedly calculates the vertical rotational angle at short intervals on the basis of the second sensing information, and calculates the difference between the time t23 and the time t24 as the stance phase of the right foot. The estimation apparatus 1 may calculate a statistical value such as the average of the time differences between the time t23 and the time t24 in each motion cycle of the walking motion as the stance phase of the right foot.

More specifically, the left foot stance phase calculation unit 141 of the estimation apparatus 1 sequentially compares the values of the vertical rotational angle calculated based on the values of the acceleration and the angular velocity included in the repeatedly acquired first sensing information, and identifies the time t13 at which the vertical rotational angle indicates a positive peak. Moreover, the left foot stance phase calculation unit 141 sequentially compares the values of the vertical rotational angle calculated based on the values of the acceleration and the angular velocity included in the repeatedly acquired first sensing information, and identifies the time t14 at which the vertical rotational angle indicates a negative peak. The left foot stance phase calculation unit 141 calculates the stance phase T1 of the left foot, which indicates the difference between the time t13 and the time t14. Similarly, the left foot stance phase calculation unit 141 calculates the stance phase T1 of the left foot for each motion cycle of the walking motion of the user. The left foot stance phase calculation unit 141 sequentially outputs the calculated stance phase T1 of the left foot to the risk estimation unit 13.

Similarly, the right foot stance phase calculation unit 142 of the estimation apparatus 1 sequentially compares the values of the vertical rotational angle calculated based on the values of the acceleration and the angular velocity included in the repeatedly acquired second sensing information, and identifies the time t23 at which the vertical rotational angle indicates a positive peak. Moreover, the right foot stance phase calculation unit 142 sequentially compares the values of the vertical rotational angle calculated based on the values of the acceleration and the angular velocity included in the repeatedly acquired second sensing information, and identifies the time t24 at which the vertical rotational angle indicates a negative peak. The right foot stance phase calculation unit 142 calculates the stance phase T2 of the right foot, which indicates the difference between the time t23 and the time t24. Similarly, the right foot stance phase calculation unit 142 calculates the stance phase T2 of the right foot for each motion cycle of the walking motion of the user. The right foot stance phase calculation unit 142 sequentially outputs the calculated stance phase T2 of the right foot to the risk estimation unit 13.

Subsequent processing of the risk estimation unit 13 is the same as that of the above-described example embodiment. It should be noted that as in the first example embodiment, the first sensor apparatus 2 may calculate the stance phase T1 of the left foot stance phase calculation unit 141 using the vertical rotational angle. Similarly, the second sensor apparatus 3 may calculate the stance phase T2 of the right foot stance phase calculation unit 142 using the vertical rotational angle.

### Fourth Example Embodiment

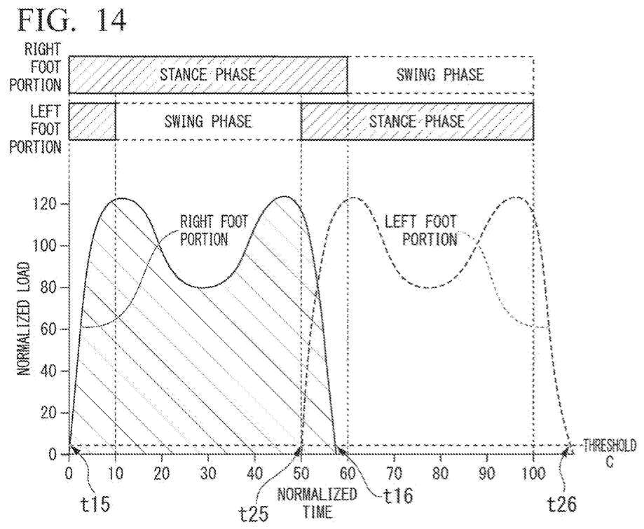

FIG. 13 is a diagram showing an outline of the sensor apparatus provided on a shoe sole according to the fourth example embodiment.

As shown in FIG. 13, the first sensor apparatus 2 and the second sensor apparatus 3 are provided in the soles of the respective shoes, and in the fourth example embodiment, pressure-sensitive sensors 1201 and 1202 are respectively provided in the vicinity of the heel and the vicinity of the toe of the shoe sole of the left foot, and pressure-sensitive sensors 1301 and 1302 are respectively provided in the vicinity of the heel and the vicinity of the toe of the shoe sole of the right foot. The first sensor apparatus 2 transmits sensing information including the pressure values obtained from the pressure-sensitive sensors 1201 and 1202 to the estimation apparatus 1. Moreover, the second sensor apparatus 3 transmits sensing information including the pressure values obtained from the pressure-sensitive sensors 1301 and 1302 to the estimation apparatus 1.

FIG. 14 is a diagram showing an outline of calculation of the stance phase according to the fourth example embodiment.

FIG. 14 shows the pressure values of each pressure-sensitive sensor in the motion cycle of the walking motion of the left foot and the right foot. When each pressure-sensitive sensor shows a pressure value equal to or greater than the threshold C, it indicates that the foot has landed.

The solid line shows the time transition of the total value of the pressure values obtained from the pressure-sensitive sensor 1301 and the pressure-sensitive sensor 1302 of the right foot. Time t15 indicates the timing at which the pressure value of the pressure-sensitive sensor 1301 at the heel of the right foot increases and the total value exceeds the threshold C. Moreover, the time t16 indicates the timing at which the pressure value of the pressure-sensitive sensor 1302 at the toe of the right foot decreases and the total value falls below the threshold C. Then, the estimation apparatus 1 repeatedly calculates the total value of the pressure values at short intervals on the basis of the first sensing information, and calculates the difference between the time t15 and the time t16 as the stance phase of the right foot. The estimation apparatus 1 may calculate a statistical value such as the average of the time differences between the time t15 and the time t16 in each motion cycle of the walking motion as the stance phase of the right foot.

The broken line shows the time transition of the total value of the pressure values obtained from the pressure-sensitive sensor 1201 and the pressure-sensitive sensor 1202 of the left foot. Time t25 indicates the timing at which the pressure value of the pressure-sensitive sensor 1201 at the heel of the left foot increases and the total value exceeds the threshold C. Moreover, the time t26 indicates the timing at which the pressure value of the pressure-sensitive sensor 1202 at the toe of the left foot decreases and the total value falls below the threshold C. Then, the estimation apparatus 1 repeatedly calculates the total value of the pressure values at short intervals on the basis of the second sensing information, and calculates the difference between the time t25 and the time t26 as the stance phase of the left foot. The estimation apparatus 1 may calculate a statistical value such as the average of the time differences between the time t25 and the time t26 in each motion cycle of the walking motion as the stance phase of the left foot.

More specifically, the left foot stance phase calculation unit 141 of the estimation apparatus 1 sequentially compares the total value of the pressure values of the heel and the toe included in the repeatedly acquired first sensing information, and identifies the time t25 at which the total value reaches the threshold C and the time t26 at which the total value falls below the threshold C from a state of having exceeded the threshold C. The left foot stance phase calculation unit 141 calculates the stance phase T1 of the left foot, which indicates the difference between the time t25 and the time t26. Similarly, the left foot stance phase calculation unit 141 calculates the stance phase T1 of the left foot for each motion cycle of the walking motion of the user. The left foot stance phase calculation unit 141 sequentially outputs the calculated stance phase T1 of the left foot to the risk estimation unit 13.

Similarly, the right foot stance phase calculation unit 142 of the estimation apparatus 1 sequentially compares the total value of the pressure values of the heel and the toe included in the repeatedly acquired second sensing information, and identifies the time t15 at which the total value reaches the threshold C and the time t16 at which the total value falls below the threshold C from a state of having exceeded the threshold C. The right foot stance phase calculation unit 142 calculates the stance phase T2 of the right foot, which indicates the difference between the time t15 and the time t16. Similarly, the right foot stance phase calculation unit 142 calculates the stance phase T2 of the right foot for each motion cycle of the walking motion of the user. The right foot stance phase calculation unit 142 sequentially outputs the calculated stance phase T2 of the right foot to the risk estimation unit 13.

Subsequent processing of the risk estimation unit 13 is the same as that of the above-described example embodiment. It should be noted that as in the first example embodiment, the first sensor apparatus 2 may calculate the stance phase T1 of the left foot stance phase calculation unit 141 using the pressure values of the sole of the foot. Similarly, the second sensor apparatus 3 may calculate the stance phase T2 of the right foot stance phase calculation unit 142 using the pressure values of the sole of the foot.

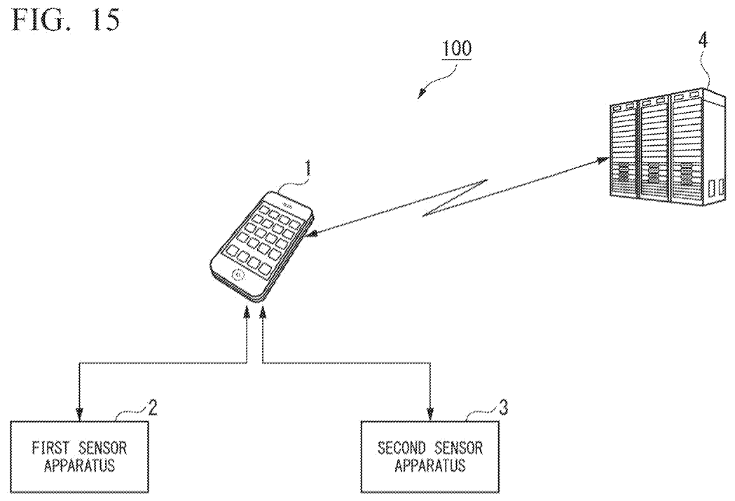

FIG. 15 is a diagram showing a schematic configuration of a lower limb abnormality risk determination system according to another example embodiment.

The lower limb abnormality risk determination system 100 may further include a server apparatus 4, and the server apparatus 4 may perform part of the processing of the estimation apparatus 1 described above. That is, the server apparatus 4 may perform at least one of the stance phase calculation process and the lower limb abnormality risk estimation process described for the estimation apparatus 1 described above. In this case, the server apparatus 4 receives the information for performing the processing via the estimation apparatus 1 and returns the processing result to the estimation apparatus 1. Then, the estimation apparatus 1 outputs the result of risk estimation of the lower limb abnormality on the basis of the information returned from the server apparatus 4.

FIG. 16 is a diagram showing the minimum configuration of the estimation apparatus.

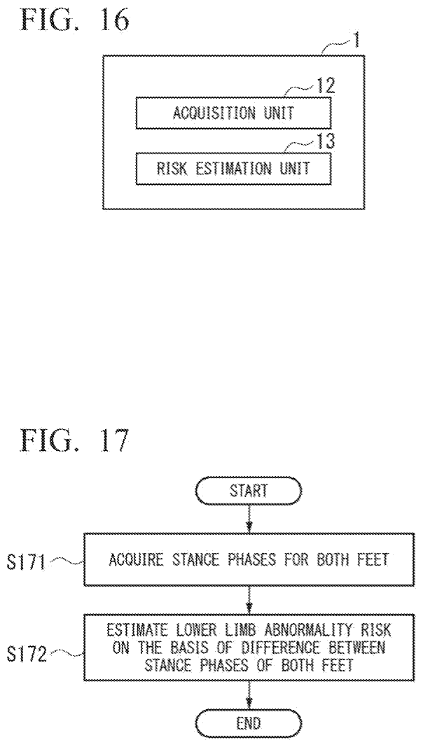

FIG. 17 is a diagram showing a processing flow of the estimation apparatus with the minimum configuration.

The estimation apparatus 1 includes at least the acquisition unit 12 and the risk estimation unit 13.

The acquisition unit 12 acquires the stance phase for each of both feet (step S171).

The risk estimation unit 13 estimates the lower limb abnormality risk on the basis of the difference in the stance phase of both feet (step S172).

Each of the above apparatuses has a computer system inside. The process of each process described above is stored in a computer-readable recording medium in the form of a program, and the above process is performed by a computer reading and executing this program. Here, the “computer-readable recording medium” refers to a magnetic disk, a magneto-optical disk, a compact disc (CD)-ROM, a digital versatile disc (DVD)-ROM, a semiconductor memory, or the like. Moreover, this computer program may be delivered to a computer via a communication line, and the computer receiving the delivery may execute the program.

Moreover, the above program may be a program for realizing some of the above-described functions. Furthermore, the program may be a so-called differential file (differential program) that can realize the above-described functions in combination with a program already recorded in the computer system.

While the present invention has been particularly shown and described with reference to example embodiments thereof, the present invention is not limited to these example embodiments. It will be understood by those of ordinary skill in the art that various changes in form and details may be made therein without departing from the spirit and scope of the present invention as defined by the claims.

What is claimed is:

1. An estimation device comprising:
a memory storing instructions; and
a processor connected to the memory and configured to execute the instructions to:
acquire sensor data including acceleration data on a vertical axis measured by sensors mounted in shoes of a user;
calculate a time of a stance phase of a left foot of the user indicating a time difference between a timing at which the acceleration data of the left foot exceeds a first threshold and a timing at which the acceleration data of the left foot exceeds a second threshold;
calculate a time of a stance phase of a right foot of the user indicating a time difference between a timing at which the acceleration data of the right foot exceeds the first threshold and a timing at which the acceleration data of the right foot exceeds the second threshold;
calculate an asymmetry index of a stance phase indicating a difference between the time of the stance phase of the left foot and the time of the stance phase of the right foot; and
estimate a lower limb abnormality risk of the user based on the asymmetry index of the stance phase.

2. The estimation device according to claim 1, wherein the sensor data is measured by the sensors placed below arches of the user's left foot and right foot.

3. The estimation device according to claim 1, wherein the processor is configured to execute the instructions to
estimate a disturbance in accordance with a time indicated by the asymmetry index by using a machine learning model generated by a machine learning using data sets of the time indicated by the asymmetry index and an actual occurrence event of the disturbance according to the time indicated by the asymmetry index.

4. The estimation device according to claim 1, wherein the processor is configured to execute the instructions to
estimate a level of the lower limb abnormality risk in accordance with a time indicated by the asymmetry index by using a machine learning model generated by a machine learning using data sets of the time indicated by the asymmetry index and the level of the lower limb abnormality risk according to the time indicated by the asymmetry index.

5. The estimation device according to claim 1, wherein the processor is configured to execute the instructions to
calculate the asymmetry index of stance phases indicating the difference between the time of the stance phase of the left foot and the time of the stance phase of the right foot.

6. The estimation device according to claim 1, wherein the processor is configured to execute the instructions to
display information regarding the estimated risk of lower limb abnormality of the user on the screen of a mobile terminal.

7. The estimation device according to claim 6, wherein the processor is configured to execute the instructions to
display information regarding the estimated risk of lower limb abnormality of the user on the screen of the mobile terminal with content optimized for healthcare application.

8. An estimation system comprising:
the estimation device according to claim 1; and
sensors that measure sensor data including acceleration data on a vertical axis.

9. An estimation method executed by a computer, the method comprising:
acquiring sensor data including acceleration data on a vertical axis measured by sensors mounted in shoes of a user;
calculating a time of a stance phase of a left foot of the user indicating a time difference between a timing at which the acceleration data of the left foot exceeds a first threshold and a timing at which the acceleration data of the left foot exceeds a second threshold;
calculating a time of a stance phase of a right foot of the user indicating a time difference between a timing at which the acceleration data of the right foot exceeds the first threshold and a timing at which the acceleration data of the right foot exceeds the second threshold;
calculating an asymmetry index of a stance phase indicating a difference between the time of the stance phase of the left foot and the time of the stance phase of the right foot; and
estimating a lower limb abnormality risk of the user based on the asymmetry index of the stance phase.

10. A non-transitory program recording medium recorded with a program causing a computer to perform the following processes:
acquiring sensor data including acceleration data on a vertical axis measured by sensors mounted in shoes of a user;
calculating a time of a stance phase of a left foot of the user indicating a time difference between a timing at which the acceleration data of the left foot exceeds a first threshold and a timing at which the acceleration data of the left foot exceeds a second threshold;
calculating a time of a stance phase of a right foot of the user indicating a time difference between a timing at which the acceleration data of the right foot exceeds the first threshold and a timing at which the acceleration data of the right foot exceeds the second threshold;
calculating an asymmetry index of a stance phase indicating a difference between the time of the stance phase of the left foot and the time of the stance phase of the right foot; and
estimating a lower limb abnormality risk of the user based on the asymmetry index of the stance phase.
